(19) United States
(12) Patent Application Publication
(10) Pub. No.: US 2011/0245179 A1
(43) Pub. Date: Oct. 6, 2011

(54) Composition for Prevention or Treatment of Heart Failure

(76) Inventors: Woo Jin Park, Gwangju (KR); Jae Gyun Oh, Gwangju (KR)

(21) Appl. No.: 13/119,328

(22) PCT Filed: Sep. 17, 2009
(86) PCT No.: PCT/KR2009/005307

§ 371 Date: Jun. 10, 2011

(30) Foreign Application Priority Data
Sep. 17, 2008 (KR) 10-2008-0091229

(51) Int. Cl. A61K 38/10; A61K 31/381; A61K 31/4965; A61K 31/497; A61P 9/10

(52) U.S. Cl. 514/16.4; 514/447; 514/252.12; 514/255.01; 514/254.06

(57) ABSTRACT

Provided are a composition for preventing or treating heart failure and a method for screening an agent for treating heart failure. The present disclosure demonstrates for the first time that administration of PKCζ inhibitor provides inotropic effect by increasing myocardial contractility. Thus, the present disclosure will contribute greatly to the prevention or treatment of heart failure. Also, since the present disclosure is based on the change in calcium sensitivity in cardiac myocytes unlike the existing inotropic agents, it can enhance the myocardial contractility without increasing oxygen demand or the risk of arrhythmia.

### CROSS-REFERENCE TO RELATED APPLICATIONS

This application claims priority under 35 U.S.C. §119 to Korean Patent Application No. 10-2008-0091229, filed on Sep. 17, 2010 in the Korean Intellectual Property Office, the disclosure of which is incorporated herein by reference in its entirety.

### TECHNICAL FIELD

The present disclosure relates to a composition for preventing or treating heart failure and a method for screening an agent for treating heart failure.

### BACKGROUND

Heart failure refers to inability of the heart to supply sufficient blood flow to meet the body's needs. It is the final and fatal form of various heart diseases, including cardiac hypertrophy, coronary arteriosclerosis, myocardial infarction, valvular heart disease, hypertension, cardiomyopathy, or the like¹. At early stages, heart failure shows reduced ability of exercise. As it progresses, the heart's capacity to supply blood declines rapidly, thus resulting in insufficient blood supply and fatal conditions such as heart attack².

Heart failure is one of the most common health problems, with a high fatality rate of 3 in 1,000 people every year. The fatality of heart failure has already exceeded that of infectious diseases and is expected to be the highest among all diseases by 2030³. In the United States, heart failure accounts for 44% of all deaths⁴. According to a recent study, it also caused the most deaths in England⁵.

Heart failure is characterized by reduced contractility of the heart muscles, thinning of the ventricular walls, and expansion of the atria and the ventricles. Although the effect of the contractility of the heart muscles on the onset of heart failure is unclear, it is known through many researches that the reduction of the myocardial contractility is closely related to the onset of heart failure^{2,6}. Accordingly, many researchers have attempted to treat heart failure using an inotropic agent that enhances the myocardial contractility. In the last decade, various inotropic agents have been tried for the treatment of heart failure. However, an inotropic agent that can completely cure heart failure has not been found yet. On the contrary, continued use of inotropic agents has aggravated symptoms⁷. Nevertheless, due to the positive results for inotropic therapy in experiments with rodents, myocardial contractility is still viewed as an attractive therapeutic target for heart failure⁸. Thus, a new type of inotropic agent that can solve the problems of the existing inotropic agents is keenly needed.

Protein kinase C (PKC)-interaction cousin of thioredoxin (PICOT), which has long been studied by the inventors of the present disclosure, has an effect of inhibiting cardiac hypertrophy and enhancing myocardial contractility⁹. Experiments with PICOT transgenic mice and PICOT gene-silenced mice revealed that overexpression of the PICOT gene resulted in dramatically enhanced myocardial contractility, whereas silencing of the PICOT gene led to decrease in the degree of maximal contraction and the rate of contraction and relaxation. Also, it was found out that PICOT binds with PKCζ and inhibits the activity of PKCζ. In the present disclosure, the change in myocardial contractility while PKCζ is inhibited was measured based on this experimental result. First identified by Heagerty AM in 1996¹⁰, PKCζ is known to be involved in apoptosis in the heart and have the effect of protecting the heart. However, inotropic effect of PKCζ has never been reported, and the current study results about the inotropic effect and related mechanism associated with the PKCζ inhibitor are solely the work of the inventors of the present disclosure.

Throughout the specification, a number of publications and patent documents are referred to and cited. The disclosure of the cited publications and patent documents is incorporated herein by reference in its entirety to more clearly describe the state of the related art and the present disclosure.

### SUMMARY

The inventors of the present disclosure have made efforts to develop an effective agent for treating heart failure to treat heart failure. As a result, they have found out that protein kinase C ζ (PKCζ) may be a molecular target for heart failure treatment. That is to say, they have found out that administration of PKCζ inhibitor to cardiac myocytes leads to change in calcium sensitivity in the cells, thus providing an inotropic effect of enhancing myocardial contractility.

The present disclosure is directed to providing a composition for preventing or treating heart failure including PKCζ inhibitor as an active ingredient.

The present disclosure is also directed to providing a method for preventing or treating heart failure.

The present disclosure is also directed to providing a method for screening an agent for treating heart failure.

In one general aspect, the present disclosure provides a composition for preventing or treating heart failure including PKCζ inhibitor as an active ingredient.

In another general aspect, the present disclosure provides a method for preventing or treating heart failure including administering to a subject a PKCζ inhibitor.

Other features and aspects will be apparent from the following detailed description, the drawings, and the claims.

### BRIEF DESCRIPTION OF THE DRAWINGS

The above and other objects, features and advantages of the present disclosure will become apparent from the following description of certain exemplary embodiments given in conjunction with the accompanying drawings, in which:

FIG. 1 shows change in myocardial contractility caused by protein kinase C ζ (PKCζ) inhibitor (A shows change in cardiac myocyte shortening, B shows the degree of maximal contraction, C shows the maximal rate of contraction, and D shows the maximal rate of relaxation.);

FIG. 2 shows change in calcium concentration in cardiac myocytes caused by PKCζ inhibitor (A shows change in calcium concentration, B shows calcium concentration in the cardiac myocytes in relaxation state, C shows calcium concentration in the cardiac myocytes in contraction state, and D shows the rate of calcium removal in the cardiac myocytes following contraction.); and

FIG. 3 shows hysteresis loops showing change in myocardial contractility caused by change in calcium concentration.

### DETAILED DESCRIPTION OF EMBODIMENTS

The advantages, features and aspects of the present disclosure will become apparent from the following description of the embodiments with reference to the accompanying drawings, which is set forth hereinafter. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art. The terminology used herein is for the purpose of describing particular embodiments only and is not intended to be limiting of the example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

The inventors of the present disclosure have made efforts to develop an effective agent for treating heart failure to treat heart failure. As a result, they have found out that protein kinase C ζ (PKCζ) may be a molecular target for heart failure treatment. That is to say, they have found out that administration of PKCζ inhibitor to cardiac myocytes leads to change in calcium sensitivity in the cells, thus providing an inotropic effect of enhancing myocardial contractility.

The present disclosure provides a composition for preventing or treating heart failure comprising PKCζ inhibitor as an active ingredient. The present disclosure is based on the finding by the inventors that inhibition of PKCζ activity in cardiac myocytes provides excellent inotropic effect, unlike existing inotropic agents.

As used herein, the term “heart failure” refers to a clinical symptom in which the stroke volume of the heart decreases below a normal value and the heart fails to supply enough blood to peripheral tissues. In other words, heart failure refers to the state in which the ability of the heart to pump blood is decreased due to various causes or enough blood cannot be supplied to the body even when the heart beats normally.

As used herein, the term “PKCζ inhibitor” refers to a synthetic or natural substance that inhibits the activity of PKCζ. In a PKCζ activity assay, the presence of the PKCζ inhibitor results in a greatly statistically significant difference in PKCζ activity as compared to its absence. For example, the presence of the PKCζ inhibitor in a PKCζ activity assay may result in inhibited phosphorylation of a synthetic or natural substance, which is a substrate of PKCζ. In addition to a substance that inhibits the activity of the PKCζ enzyme, the PKCζ inhibitor may also be a substance that suppresses expression of the PKCζ gene.

In case the PKCζ inhibitor inhibits the activity of the enzyme, the composition of the present disclosure may include antibody, peptide, chemical or natural extract as an active ingredient.

The antibody that may be used in the present disclosure is a polyclonal or monoclonal antibody, specifically a monoclonal antibody, that specifically binds to the PKCζ protein and inhibits its activity. The antibody to the PKCζ protein may be prepared according to methods commonly employed in the art, for example, fusion method (Kohler and Milstein, European Journal of Immunology, 6: 511-519 (1976)), recombinant DNA method (U.S. Pat. No. 4,816,567) or phage antibody library method (Clackson et al, Nature, 352: 624-628 (1991); Marks et al, J. Mol. Biol., 222: 58, 1-597 (1991)). General procedures for producing antibody are described in detail in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Fla., 1984; and Coligan, Current Protocols in Immunology, Wiley/Greene, NY, 1991, which are incorporated herein by references. For example, the preparation of hybridoma cells for monoclonal antibody production may be done by fusion of an immortal cell line and the antibody-producing lymphocytes, which can be achieved easily by techniques well known in the art. Polyclonal antibodies may be prepared by injecting PKCζ protein antigen to a suitable animal, collecting antiserum from the animal, and isolating antibodies employing a known affinity technique.

As used herein, the term “natural extract” refers to an extract obtained from various organs or parts (e.g., leaves, flowers, roots, stems, branches, peel, fruits, etc.) of a natural source. The natural extract may be obtained using (a) water, (b) C₁-C₄ absolute or hydrated alcohol (e.g., methanol, ethanol, propanol, butanol, n-propanol, isopropanol, n-butanol, etc.), (c) mixture of the lower alcohol with water, (d) acetone, (e) ethyl acetate, (f) chloroform, (g) 1,3-butylene glycol, (h) hexane, or (i) diethyl ether as an extraction solvent.

Further, the natural extract includes, in addition to those obtained from solvent extraction, ones produced by common purification processes. For example, the natural extract includes the fractions obtained through various additional purification processes, such as separation using an ultrafiltration membrane having a predetermined molecular weight cut off, separation by various chromatographic techniques (based on size, charge, hydrophobicity or affinity), or the like. The natural extract may be prepared into powder through additional processes such as vacuum distillation, lyophilization or spray drying.

In case the PKCζ inhibitor inhibits gene expression, the composition of the present disclosure may comprise an antisense or siRNA oligonucleotide as the active ingredient.

As used herein, the term “antisense oligonucleotide” refers to a DNA, an RNA or a derivative thereof including a nucleotide sequence complementary to a specific mRNA sequence, thus binding to the complementary sequence of the mRNA and inhibiting translation of the mRNA into a protein. The antisense sequence is a DNA or RNA sequence complementary to PKCζ mRNA and capable of binding to the PKCζ mRNA, thus inhibiting translation of the PKCζ mRNA, translocation into the cytoplasm, maturation, or any other activity essential to overall biological functions. The antisense nucleotide may be 6 to 100 bases long, specifically 8 to 60 bases long, more specifically 10 to 40 bases long.

The antisense nucleotide may be modified at one or more base, sugar or backbone positions to improve the desired effect (De Mesmaeker et al., Curr Opin Struct Biol., 5(3): 343-55 (1995)). For example, the nucleotide backbone may be modified with phosphorothioate, phosphotriester, methylphosphonate, single-chain alkyl, cycloalkyl, single-chain heteroatomic, or heterocyclic sugar-sugar bonding. Also, the antisense nucleotide may include one or more substituted sugar moiety. The antisense nucleotide may include a modified base. The modified base may include hypoxanthine, 6-methyladenine, 5-methylpyrimidine (especially, 5-methylcytosine), 5-hydroxymethylcytosine (HMC), glycosyl HMC, gentiobiosyl HMC, 2-aminoadenine, 2-thiouracil, 2-thiothymine, 5-bromouracil, 5-hydroxymethyluracil, 8-azaguanine, 7-deazaguanine, N6-(6-aminohexyl)adenine, 2,6-diaminopurine, etc. Also, the antisense nucleotide may be chemically bonded to one or more moiety or conjugate that improves activity and cell attachment of the antisense nucleotide. The moiety may be an oil-soluble moiety, such as cholesterol moiety, cholesteryl moiety, cholic acid, thioether, thiocholesterol, aliphatic chain, phospholipid, polyamine, polyethylene glycol chain, adamantane acetic acid, palmityl moiety, octadecylamine, and hexylamino-carbonyl-oxycholesterol moiety, but is not limited thereto. Methods for preparing oligonucleotides having oil-soluble moieties are well known in the related art (U.S. Pat. Nos. 5,138,045, 5,218,105 and 5,459,255). The modified nucleotide may have provide increased stability against nucleases and improved binding ability of the antisense nucleotide to the target mRNA.

The antisense oligonucleotide may be either synthesized in vitro and administered into the body or it may be synthesized in vivo. An example of synthesizing the antisense oligonucleotide in vitro is to use RNA polymerase I. An example of synthesizing the antisense oligonucleotide in vivo is to use a vector having the origin of the multiple cloning site (MCS) in opposite direction so that the antisense RNA is transcribed. Specifically, the antisense RNA may have a translation stop codon within its sequence in order to prevent translation into a peptide sequence.

As used herein, the term “siRNA” refers to a nucleotide molecule capable of mediating RNA interference (RNAi) or gene silencing (see WO 00/44895, WO 01/36646, WO 99/32619, WO 01/29058, WO 99/07409 and WO 00/44914). Since siRNA can suppress the expression of the target gene, it provides an effective way of gene knockdown or genetic therapy. First discovered in plants, worms, fruit flies and parasites, siRNA has been recently developed and used for studies of mammal cells.

In case the siRNA molecule is used in the present disclosure, it may have a structure in which its sense strand (a sequence corresponding to the PKCζ mRNA sequence) and its antisense strand (a sequence complementary to the PKCζ mRNA sequence) form a double strand. Alternatively, it may have a single-stranded structure having self-complementary sense and antisense strands.

The siRNA is not limited to those in which double-stranded RNA moieties constitute complete pairs, but includes the unpaired moieties such as mismatch (corresponding bases are not complementary), bulge (no base in one chain), etc. The total length of the siRNA may be 10 to 100 bases, specifically 15 to 80 bases, more specifically 20 to 70 bases.

The end of the siRNA may be either blunt or cohesive as long as it is capable of suppressing the expression of the PKCζ gene via RNAi. The cohesive end may be either 3′- or 5′-end.

In the present disclosure, the siRNA molecule may have a short nucleotide sequence (e.g., about 5-15 nucleotides) inserted between the self-complementary sense and antisense strands. In this case, the siRNA molecule formed from the expression of the nucleotide sequence forms a hairpin structure via intramolecular hybridization, resulting in a stem-and-loop structure overall. The stem-and-loop structure is processed in vitro or in vivo to give an activated siRNA molecule capable of mediating RNAi.

In a specific embodiment of the present disclosure, the PKCζ inhibitor used in the composition of the present disclosure as the active ingredient is a substance inhibiting the enzymatic activity of PKCζ.

In a specific embodiment of the present disclosure, the PKCζ inhibitor is a compound of Chemical Formula I:

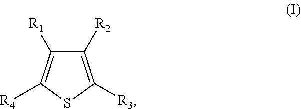

wherein each of R₁ and R₂ is independently alkoxycarbonyl, substituted alkoxycarbonyl, aryl or substituted aryl, wherein at least one of R₁ and R₂ is alkoxycarbonyl or substituted alkoxycarbonyl, and at least one of R₁ and R₂ is aryl or substituted aryl; and each of R₃ and R₄ is independently H, C₁-C₃ alkyl, substituted C₁-C₃alkyl or NHR₅, wherein R₅ is H,

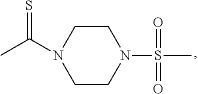

acyl or substituted acyl, and at least one of R₃ and R₄ is NHR₅.

As used herein, the term “alkoxycarbonyl” refers to the C(O)OR₆ group, wherein R₆ is a C₁-C₄ straight, branched, substituted straight or substituted branched group. For example, it includes methoxycarbonyl, ethoxycarbonyl, tert-butoxycarbonyl, isobutoxycarbonyl, n-butoxycarbonyl, propoxycarbonyl and isopropoxycarbonyl.

As used herein, the term “aryl” refers to a monocyclic or bicyclic aromatic hydrocarbon ring having 6-12 carbon atoms in the ring. The monocyclic or bicyclic aromatic hydrocarbon may be a heterocyclic ring having one or more heteroatoms such as S, O, N or P. For example, phenyl, naphthalenyl, piperazinyl, biphenyl and diphenyl are included.

As used herein, the term “substituted aryl” refers to an aryl group having a substituent at any possible position.

As used herein, the term “substituted alkoxycarbonyl” refers to an alkoxycarbonyl group having a substituent at any possible position.

As used herein, the term “substituted C₁-C₃ alkyl” refers to a C₁-C₃ alkyl group having a substituent at any possible position.

As used herein, the term “substituted acyl” refers to an acyl group having a substituent at any possible position. For example, the substituent may include alkyl, substituted alkyl, hydroxyalkylthio, alkylsulfonyl, alkylsulfinyl, alkoxy, alkoxyalkyl, alkoxycarbonyl, alkoxyarylthio, alkoxycarbonyl, alkylcarbonyloxy, aryl, aryloxy, arylalkyl, arylalkyloxy, arylsulfinyl, arylsulfinylalkyl, arylsulfonylaminocarbonyl, alkanoyl, substituted alkanoyl, alkanoylamino, alkylcarbonyl, aminocarbonylaryl, aminocarbonylalkyl, arylazo, alkoxycarbonylalkoxy, arylcarbonyl, alkylaminocarbonyl, aminoalkylcarbonyl, arylaminocarbonyl, alkylcarbonyloxy, alkylcarbonylamino, arylcarbonylamino, arylsulfonyl, alkenyl, substituted alkenyl, alkynyl, substituted alkynyl, amino, substituted amino, aminoalkyl, substituted aminoalkyl, alkylamino, substituted alkylamino, doubly substituted amino, aminocarbonyl, arylamino, arylalkylamino, arylalkoxy, arylalkylthio, cyano, cycloalkyl, substituted cycloalkyl, cycloalkylalkyl, cycloalkylalkoxy, carboxyl, substituted carboxyl, carboxyalkyl, carboxyalkoxy, carbamoyl, halogen, haloalkyl, haloalkoxy, heterocycloalkyl, substituted heterocycloalkyl, heterocycloalkylalkyl, heteroaryl, substituted heteroaryl, heteroarylthio, heteroaryloxy, heteroarylalkenyl, heteroarylheteroaryl, heteroarylalkylthio, heteroaryloxyalkyl, heteroarylsulfonyl, heterocycloalkylsulfonyl, nitro, sulfonyl, sulfonamide, substituted sulfonamide, thio, thioalkyl and ureido.

In a specific embodiment of the present disclosure, the PKCζ inhibitor is a compound selected from the compounds of Chemical Formulas II to VI or a combination thereof:

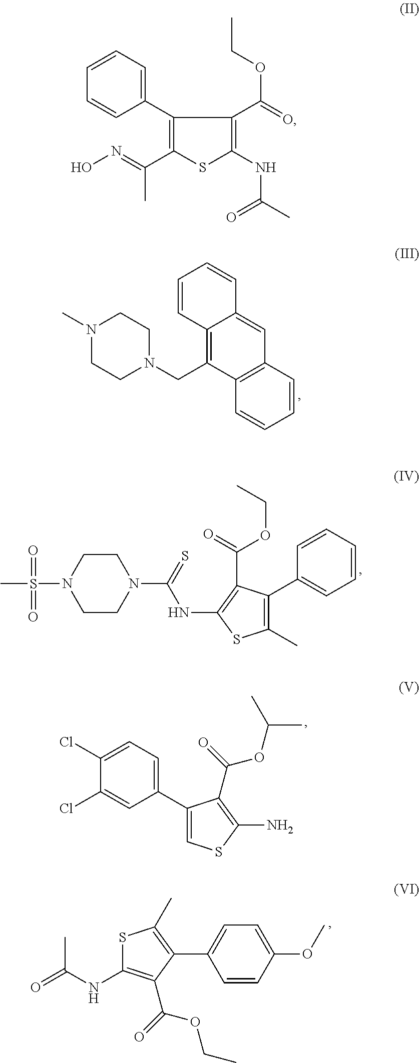

The compound of Chemical Formula II is ethyl (5E)-2-acetylimino-5-[1-(hydroxyamino)ethylidene]-4-phenyl-thiophene-3-carboxylate. It has an IC₅₀ value of 10 μM for PKCζ, whereas it has an IC₅₀ value of more than 100 μM for PKCδ or PKCβ.

The compound of Chemical Formula III is 1-(anthracen-9-ylmethyl)-4-methyl-piperazine. It has an IC₅₀ value of 25 μM for PKCζ, whereas it has an IC₅₀ value of more than 100 μM for PKCδ and 50 μM for PKCβ.

The compound of Chemical Formula IV provides an inhibitory effect of about 1.2 times that of the compound of Chemical Formula II when tested at 100 μM. The compound of Chemical Formula V provides an inhibitory effect of about 1.8 times that of the compound of Chemical Formula II when tested at 100 μM. And, the compound of Chemical Formula V provides an inhibitory effect of about 2.6 times that of the compound of Chemical Formula II when tested at 100 μM (see US patent Application No. 20080021036).

In a specific embodiment of the present disclosure, the PKCζ inhibitor is a compound of Chemical Formula VII:

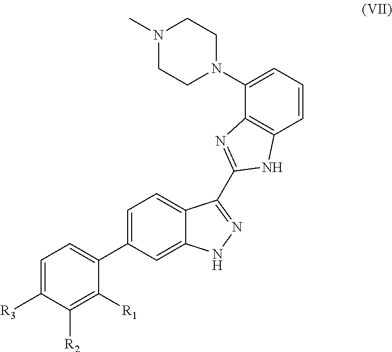

In the above formula, R₁ is hydrogen or C₁-C₁₀ alkoxy (specifically C₁-C₅ alkoxy, more specifically C₁-C₃ alkoxy, and most specifically methoxy), R₂ is hydrogen, halo (specifically F, Cl, Br or I, more specifically F or Cl, and most specifically F), amine or C₁-C₁₀ alkoxy (specifically C₁-C₅ alkoxy, more specifically C₁-C₃ alkoxy, and most specifically methoxy), and R₃ is hydrogen, hydroxy, halo (specifically F, Cl, Br or I, more specifically F or Cl, and most specifically F), amine, carboxyl, C₁-C₅ alkylamine (specifically C₁-C₃ alkyl amine, and most specifically methylamine), C₁-C₅ alcohol (specifically methanol, ethanol or propanol, and most specifically methanol), C₁-C₁₀ alkoxy (specifically C₁-C₅ alkoxy, more specifically C₁-C₃ alkoxy, and most specifically methoxy), —NHCO—R₄ (R₄ is C₁-C₅ alkyl, specifically methyl, ethyl or propyl, and most specifically methyl), —NH—R₅ (R₅ is C₁-C₅ alkyl, specifically methyl, ethyl or propyl, and most specifically methyl), —N(R₆)₂ (R₆ is C₁-C₃ alkyl, specifically methyl), —CO—R₇ (R₇ is C₁-C₅ alkyl, specifically methyl, ethyl or propyl, and most specifically methyl), —CONH₂ or —SO₂NH₂.

In a specific embodiment of the present disclosure, the PKCζ inhibitor is a compound of Chemical Formula VIII:

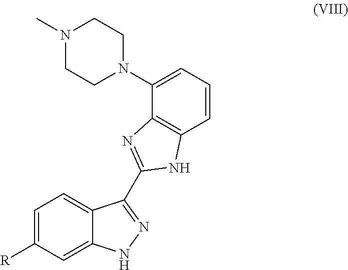

wherein R is indolyl, quinolyl, indazole or benzofuran.

Specific examples of the PKCζ inhibitor used in the present disclosure are the compounds of Chemical Formulas IX and X:

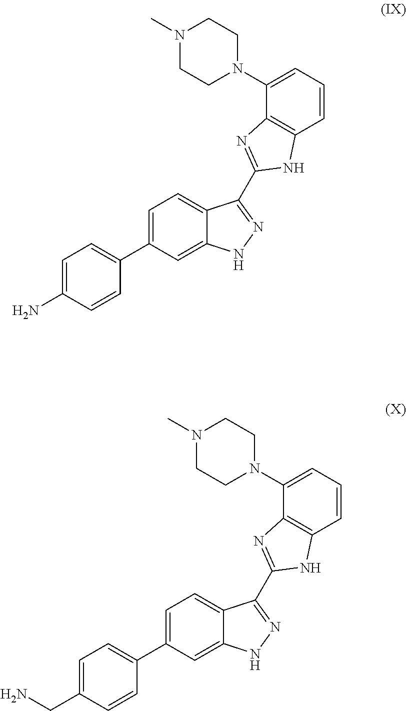

Most specifically, the PKCζ inhibitor may be the compound of Chemical Formula IX.

In a specific embodiment of the present disclosure, the PKCζ inhibitor is a peptide comprising an amino acid sequence of SEQ ID NO: 1 or 2.

As used herein, the term “peptide” refers to a straight-chain molecule consisting of amino acid residues linked by peptide bonds. It may consist of 4-40, specifically 4-30, most specifically 4-20, amino acid residues.

The PKCζ inhibitor peptide of the present disclosure is prepared according to the solid-phase synthesis technique commonly employed in the art (Merrifield, R. B., J. Am. Chem. Soc., 85: 2149-2154 (1963), Kaiser, E., Colescot, R. L., Bossinger, C. D., Cook, P. I., Anal. Biochem., 34: 595-598 (1970)). That is to say, amino acids with α-amino and side-chain groups protected are attached to a resin. Then, after removing the α-amino protecting groups, the amino acids are successively coupled to obtain an intermediate. The amino acid sequence for preparing the PKCζ inhibitor peptide of the present disclosure may be referred to in the existing techniques (Chen L, Hahn H, Wu G, Chen C H, Liron T, Schechtman D, Cavallaro G, Banci L, Guo Y, Bolli R, Dorn G W, Mochly-Rosen D., Proc. Natl. Acad. Sci., 98, 11114-9 (2001); Phillipson A, Peterman E E, Taormina P Jr, Harvey M, Brue R J, Atkinson N, Omiyi D, Chukwu U, Young L H., Am. J. Physiol. Heart Circ. Physiol., 289, 898-907 (2005); and Wang J, Bright R, Mochly-Rosen D, Giffard R G., Neuropharmacology., 47, 136-145 (2004)).

In a specific embodiment of the present disclosure, the peptide is further bonded to a membrane-permeable peptide.

For the PKCζ inhibitor peptide of the present disclosure to be transferred into a cardiac myocyte, it should contain the membrane-permeable peptide. As used herein, the term “membrane-permeable peptide” refers to a peptide necessarily required to transfer a specific peptide into a cell. Usually, it consists of 10-50 or more amino acid sequences.

The membrane-permeable peptide is a peptide capable of passing through the phospholipid bilayer of the cell membrane as it is. For example, it includes a Tat-derived peptide, a signal peptide (e.g., a cell-penetrating peptide), an arginine-rich peptide, a transportan, or an amphiphipathic peptide carrier, but without being limited thereto (Morris, M. C. et al., Nature Biotechnol. 19: 1173-1176 (2001); Dupont, A. J. and Prochiantz, A., CRC Handbook on Cell Penetrating Peptides, Langel, Editor, CRC Press (2002); Chaloin, L. et al., Biochemistry 36(37): 11179-87 (1997); and Lundberg, P. and Langel, U., J. Mol. Recognit. 16(5): 227-233 (2003)). In addition to these naturally occurring peptides, various antennapedia-based peptides capable of crossing the cell membrane are known, including retroinverso and D-isomer peptides (Brugidou, J. et al., Biochem Biophys Res Commun. 214(2): 685-93 (1995); Derossi, D. et al., Trends Cell Biol. 8: 84-87 (1998)).

Most specifically, the Tat-derived peptide may be used as the membrane-permeable peptide.

The Tat protein, which originates from human immunodeficiency virus (HIV), consists of 86 amino acids and has cysteine-rich, basic and integrin-binding domains as major protein domains. Although the Tat peptide has a cell membrane-penetrating property only with the YGRKKRRQRRR (i.e., the 48th to 60th amino acids) sequence, it is known that a more efficient penetration is possible when it has a branched structure including several copies of the RKKRRQRRR sequence (Tung, C. H. et al., Bioorg. Med. Chem. 10: 3609-3614 (2002)). The various Tat peptides having cell membrane-penetrating ability are described in Schwarze, S. R. et al., Science 285: 1569-1572 (1999).

In a specific embodiment of the present disclosure, an adequate concentration of the PKCζ inhibitor peptide to inhibit the PKCζ protein in cardiac myocytes is 300-700 nM, specifically 400-600 nM, most specifically 500 nM.

In a specific embodiment of the present disclosure, the composition of the present disclosure may be prepared as a pharmaceutical composition or a food composition.

If the composition of the present disclosure is a pharmaceutical composition, the composition includes: (i) an effective amount of the PKCζ inhibitor peptide of the present disclosure; and (ii) a pharmaceutically acceptable carrier. As used herein, the term “effective amount” means an amount sufficient to exert the above-descried therapeutic effect.

The pharmaceutically acceptable carrier included in the pharmaceutical composition of the present disclosure is one commonly used in the art and includes carbohydrate compounds (e.g., lactose, amylose, dextrose, sucrose, sorbitol, mannitol, starch, cellulose, etc.), gum acacia, calcium phosphate, alginate, gelatin, calcium silicate, microcrystalline cellulose, polyvinylpyrrolidone, cellulose, water, syrup, salt solution, alcohol, gum arabic, vegetable oils (e.g., corn oil, cottonseed oil, soybean oil, olive oil, or coconut oil), polyethylene glycol, methyl cellulose, methyl hydroxybenzoate, propyl hydroxybenzoate, talc, magnesium stearate, mineral oil, etc., but is not limited thereto. The pharmaceutical composition of the present disclosure may further include, in addition to the above ingredients, a lubricant, a wetting agent, a sweetener, a flavor, an emulsifier, a suspending agent, a preservative, or the like. Suitable pharmaceutically acceptable carriers and preparations are described in detail in Remington's Pharmaceutical Sciences (19th ed., 1995).

The pharmaceutical composition of the present disclosure may be administered orally or parenterally. Methods for parenteral administration include intravenous injection, subcutaneous injection, intramuscular injection, and the like.

An adequate administration dose of the pharmaceutical composition of the present disclosure may vary depending on various factors, such as method of preparation, method of administration, age, body weight, sex and physical conditions of the patient, diet, administration period, administration route, excretion rate, and response sensitivity. A physician of ordinary skill in the art will easily determine and diagnose an administration dose effective for the desired treatment or prevention. In a specific embodiment of the present disclosure, the adequate administration dose is 0.0001-100 mg/kg (body weight) per day. The administration can be given once or several times a day.

The pharmaceutical composition of the present disclosure may be formulated into a unit or multiple dosage form using a pharmaceutically acceptable carrier and/or excipient according to a method commonly known in the art. The formulation may be a solution in an oily or aqueous medium, a suspension or emulsion, an extract, a powder, a granule, a tablet, or a capsule. It may further include a dispersant or a stabilizer.

The composition of the present disclosure may be prepared as a food composition, particularly a functional food composition. The functional food composition of the present disclosure includes ingredients commonly used in the preparation of food. For example, it may include proteins, carbohydrates, fats, nutrients and flavoring agents. For instance, a drink may further include, in addition to the PKCζ inhibitor as the active ingredient, a flavoring agent or a natural carbohydrate. For example, the natural carbohydrate may be a monosaccharide (e.g., glucose, fructose, etc.), a disaccharide (e.g., maltose, sucrose, etc.), an oligosaccharide, a polysaccharide (e.g., dextrin, cyclodextrin, etc.), or a sugar alcohol (e.g., xylitol, sorbitol, erythritol, etc.). The flavoring agent may be a natural flavoring agent (e.g., thaumatin, stevia extract, etc.) or a synthetic flavoring agent (e.g., saccharin, aspartame, etc.).

In a specific embodiment of the present disclosure, the heart failure that may be treated by the composition of the present disclosure is induced by cardiac hypertrophy, coronary arteriosclerosis, myocardial infarction, valvular heart disease, hypertension or cardiomyopathy.

In a specific embodiment of the present disclosure, the PKCζ inhibitor enhances myocardial contractility by increasing calcium sensitivity in cardiac myocytes.

The present disclosure further provides a method for screening an agent for treating heart failure comprising: (a) contacting a sample to be analyzed with PKCζ; and (b) analyzing whether the sample binds to PKCζ or whether the sample inhibits the activity of PKCζ.

The screening method of the present disclosure may be carried out variously. Particularly, it may be performed in a high-throughput manner using various known binding assay techniques.

In the screening method of the present disclosure, the sample or the PKCζ protein may be labeled with a detectable label. For example, the detectable label may be a chemical label (e.g., biotin), an enzymatic label (e.g., horseradish peroxidase, alkaline phosphatase, peroxidase, luciferase, β-galactosidase and β-glucosidase), a radioactive label (e.g., C¹⁴, I¹²⁵, P³² and S³⁵), a fluorescent label [e.g., coumarin, fluorescein, fluorescein isothiocyanate (FITC), rhodamine 6G, rhodamine B), 6-carboxy-tetramethyl-rhodamine (TAMRA), Cy-3, Cy-5, Texas Red, Alexa Fluor, 4,6-diamidino-2-phenylindole (DAPI), HEX, TET, Dabsyl and FAM], a luminescent label, a chemiluminescent label, a fluorescence resonance energy transfer (FRET) label, or a metal label (e.g., gold and silver).

When the PKCζ protein or the sample is labeled with the detectable label, the binding between the PKCζ protein and the sample may be analyzed by detecting signals from the label. For instance, when alkaline phosphatase is used as the label, signals are detected using a chromogenic substrate such as bromochloroindolyl phosphate (BCIP), nitro blue tetrazolium (NBT), naphthol-AS-B1-phosphate or enhanced chemifluorescent (ECF) substrate. When horseradish peroxidase is used as the label, signals are detected using such substrates as chloronaphthol, aminoethylcarbazole, diaminobenzidine, D-luciferin, lucigenin (bis-N-methylacridinium nitrate), resorufin benzyl ether, luminol, Amplex Red (10-acetyl-3,7-dihydroxyphenoxazine), HYR (p-phenylenediamine-HCl and pyrocatechol), tetramethylbenzidine (TMB), 2,2′-azino-bis(3-ethylbenzthiazoline sulfonate (ABTS), o-phenylenediamine (OPD) or naphthol/pyronine.

Alternatively, the binding of the sample with the PKCζ protein may be analyzed without labeling the interactants. For example, a microphysiometer may be used to analyze whether the sample binds to the PKCζ protein. The microphysiometer is an analytical tool measuring the acidification rate of the environment of cells using a light-addressable potentiometric sensor (LAPS). The change in the acidification rate may be utilized as an indicator of the binding between the sample and the PKCζ protein (McConnell et al., Science 257: 1906-1912 (1992)).

The binding ability between the sample and the PKCζ protein may be analyzed by real-time bimolecular interaction analysis (BIA) (Sjolander & Urbaniczky, Anal. Chem. 63: 2338-2345 (1991), and Szabo et al., Curr. Opin. Struct. Biol. 5: 699-705 (1995)). BIA is the technique of analyzing specific interactions in real time and allows analysis without labeling of the interactants (e.g., BIAcore™). The change in surface plasmon resonance (SPR) may be utilized as an indicator of the real-time interactions between molecules.

Also, the screening method of the present disclosure may be performed by two-hybrid analysis or three-hybrid analysis (U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and WO 94/10300). In this case, the PKCζ protein may be used as the bait protein. Using this method, the substance that binds to the PKCζ protein, especially protein, may be screened. The two-hybrid system is based on the modular characteristics of the transcription factors consisting of splittable DNA-binding and activating domains. Briefly, this technique employs two DNA constructs. For example, in one construct, a PKCζ-encoding polynucleotide is fused with a DNA binding domain-encoding polynucleotide of a known transcription factor (e.g., GAL-4). And, in the other construct, a DNA sequence encoding the protein to be analyzed (”prey” or “sample”) is fused with a polynucleotide encoding the activating domain of the known transcription factor. When the bait and the prey interact and bind in vivo, the DNA-binding and activating domains of the transcription factor are brought in proximity and transcription of reporter genes (e.g., LacZ) occur. The detection of the expression of the reporter gene confirms that the analyte protein binds with the PKCζ protein, meaning that it can be utilized as an agent for treating or preventing heart failure.

According to the method of the present disclosure, first, the sample to be analyzed is contacted with the PKCζ protein. In the context related to the screening method of the present disclosure, the term “sample” refers to an unknown substance which is screened to test whether it affects the activity of the PKCζ protein. The sample may be a chemical, a peptide or a natural extract, but is not limited thereto. The sample analyzed by the screening method of the present disclosure may be an individual compound or a mixture of compounds (e.g., natural extract, or cell or tissue culture). The sample may be obtained from a library of synthetic or natural compounds. The method for obtaining the library of such compounds is known in the art. A library of synthetic compounds is commercially available from Maybridge Chemical Co. (UK), Comgenex (USA), Brandon Associates (USA), Microsource (USA) and Sigma-Aldrich (USA), and a library of natural compounds is commercially available from Pan Laboratories (USA) and MycoSearch (USA). The sample may be obtained through various known combinational library methods. For example, it may be acquired by a biological library method, a spatially-addressable parallel solid phase or solution phase library method, a synthetic library method requiring deconvolution, a “one-bead/one-compound” library method, and a synthetic library method using affinity chromatography selection. The methods for obtaining the molecular libraries are described in DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994, and so forth.

Subsequently, the amount or the activity of the PKCζ protein is measured in cells treated with the sample. If down-regulation of the amount or activity of the PKCζ protein is observed as the result thereof, the sample may be decided as a substance capable of treating or preventing heart failure.

In the screening method of the present disclosure, the change in the amount of the PKCζ protein may be measured by various immunoanalysis techniques known in the art. For example, the change in the amount of the PKCζ protein may be measured by radioactivity immunoanalysis, radioactive immunoprecipitation, immunoprecipitation, enzyme-linked immunosorbent assay (ELISA), capture-ELISA, inhibition or competition assay, or sandwich immunoanalysis, but without being limited thereto.

Further, the screening method of the present disclosure may be carried out by investigating whether the function of the PKCζ protein is suppressed by the sample. For example, upon treatment with a specific sample, if it is determined that the activity of the PKCζ protein is inhibited and phosphorylation of the substrate by the PKCζ protein is decreased, the tested sample is determined as suppressing the function of the PKCζ protein and thus is decided as a candidate substance for the treatment or prevention of heart failure.

### EXAMPLES

The examples and experiments will now be described. The following examples and experiments are for illustrative purposes only and not intended to limit the scope of this disclosure.

1. Experimental Methods

Management of Test Animals

Test animals were raised at an indoor temperature of 22±1° C., with 12-hour light/dark cycle. Feed and water were given freely. All the procedures followed the approved animal management guidelines and international policies.

Synthesis of and Treatment with Peptide Inhibitor

The sequence of a peptide inhibitor was designed based on the experiment of Daria Mochly-Rosen¹¹⁻¹³. The amino acid sequences of the protein kinase C ζ (PKCζ) inhibitor are described as SEQ ID NO: 1 and SEQ ID NO: 2. They are called the pseudosubstrate region. In contrast, the amino acid sequence of the PKCα inhibitor is QLVIAN. Each peptide inhibitor is linked at the amino-end with the TAT peptide YGRKKRRQRRR via the GGG bridge. As a normal group for comparison, a peptide comprising the TAT amino acid sequence was used. Cardiac myocytes isolated from 10-week-old Sprague-Dawley (SD) rat were used to test the efficiency of the peptide inhibitor. Freshly isolated cardiac myocytes were incubated along with the peptide inhibitor at 500 nM for 30 minutes in a 37° C. incubator, and then myocardial contractility was measured.

Isolation of Ventricular Cardiac Myocytes

The cardiac myocytes were isolated based on the modification of the Ren's method¹⁴. 10-week-old male SD rats (250-300 g) were used for the experiment. After injecting heparin (50 unit), the test animal was anesthetized with isoflurane and the heart was taken out immediately. The heart was connected to a pump and Tyrode's buffer [137 mM NaCl, 5.4 mM KCl, 1 mM MgCl₂, 10 mM glucose, 10 mM HEPES, 10 mM 2,3-butanedione monoxime and 5 mM taurine (Sigma), pH 7.4] at 37° C. was supplied through the coronary artery. After removing blood from the heart by pumping for 5 minutes, intercellular adhesion molecules were digested by perfusing enzyme solution [collagenase type B (0.35 U/mL, Roche), hyaluronidase (0.1 mg/mL, Sigma)] through the coronary artery. After sufficient digestion through perfusion for 20 minutes, the heart was stabilized and protected from the enzymes in 0.5% BSA solution. In all the experiments, only the rod-shaped, healthy cardiac myocytes with a distinct striation pattern were used.

Culturing of Adult Rat Cardiac Myocytes

The entire culture procedure was carried out in a class II flow hood. Culture dishes were precoated for 1 hour with 40 g/mL mouse laminin (BD Biosciences) at room temperature. The isolated cardiac myocytes were cultured in Dulbecco's minimal essential medium (HyClone) containing 50 units/mL penicillin, 50 μg/mL streptomycin, 5 mM taurine, 5 mM carnitine and 5 mM carnitine. The cardiac myocytes were stabilized for 2 hours in a 5% CO₂ incubator at 37° C., and then myocardial contractility was measured.

Measurement of Myocardial Contractility

Myocardial contractility was measured using a video-based edge detection system (IonOptix; Milton, Mass.)¹⁵. The cultured cardiac myocytes were placed on a over slip and observed under an inverted microscope (Nikon Eclipse TE-100F). To the cardiac myocytes, Tyrode's buffer (137 mM NaCl, 5.4 mM KCl, 1 mM MgCl₂, 10 mM glucose and 10 mM HEPES, pH 7.4) was continuously supplied (at 37° C., at a rate of ˜1 mL/min). The cells were stimulated with a voltage of 30 V at 1 Hz. A STIM-AT stimulator/thermostat was used. The motion of the cardiac myocytes was displayed on a computer screen by an IonOptix MyoCam camera. The motion was recorded at every 8.3 ms. The recorded motion of the cardiac myocytes was analyzed with the soft-edge software (IonOptix).

Measurement of Change in Intracellular Calcium Level

The calcium indicator Fura-2AM (Molecular Probes, USA) was added to the cardiac myocytes at a concentration of 0.5 μM for 15 minutes at 37° C. The fluorescence radiation resulting from the change in calcium level was measured using a dual-excitation single-emission fluorescence photomultiplier system (IonOptix). The cultured cardiac myocytes were placed on a over slip and observed under an inverted microscope (Nikon Eclipse TE-100F). To the cardiac myocytes, Tyrode's buffer (137 mM NaCl, 5.4 mM KCl, 1 mM MgCl₂, 10 mM glucose and 10 mM HEPES, pH 7.4) was continuously supplied (at 25° C., at a rate of ˜1 mL/min). The cells were stimulated with a voltage of 30 V at 1 Hz. A STIM-AT stimulator/thermostat was used. A 75-W halogen lamp was used as light source, and a 360 nm or 380 nm filter was used. Fluorescence of 360 and 380 nm was alternately irradiated to the cardiac myocytes. Fluorescence radiation (at 480 and 520 nm) was measured using a photomultiplier tube.

Preparation of PKCζ Inhibitor

### 2-(4-Methylpiperazin-1-yl)-6-nitroaniline (1)

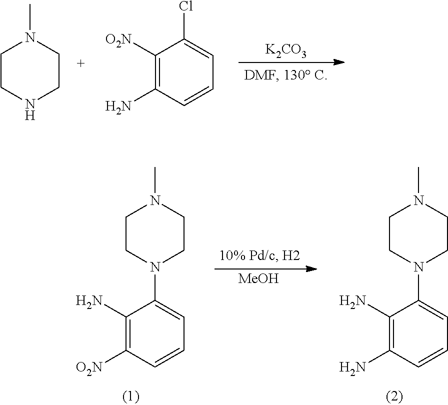

1-Mthylpiperazine (6 mL, 58.15 mmol) was dissolved in DMF (60 mL), and 3-chloro-2-nitroaniline (5 g, 28.97 mmol) and K₂CO₃ (9 g, 65.12 mmol) were added thereto. The reaction mixture was stirred at 130° C. for 12 hours. The reaction mixture was cooled with water and diluted with ethyl acetate. Upon phase separation, the organic layer was washed with brine, dried with Mg₂SO₄, filtered, and then concentrated under reduced pressure. Purification by silica gel column chromatography (ethyl acetate:hexane=3:1, MC:MeOH=10:1) yielded an orange solid substance (1, 5.8 g; yield=87.3%).

### 3-(4-Methylpiperazin-1-yl)benzene-1,2-diamine (2)

2-(4-Methylpiperazin-1-yl)-6-nitroaniline (5.8 g, 24.56 mmol) was reduced by hydrogenation for 6 hours using H₂ in the presence of 10% Pd/C in methanol (100 mL). After filtering through Celite, the solvent was removed under reduced pressure. Purification by silica gel column chromatography (MC:MeOH=5:1) yielded a grey solid substance (2, 5.2 g; yield=85.3%).

### 6-Bromo-1H-indazole-3-carbaldehyde (3)

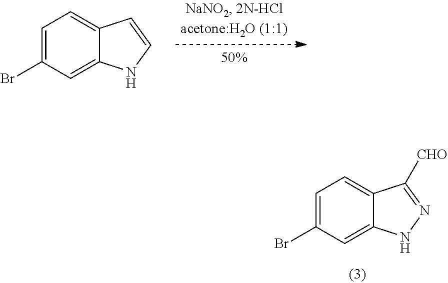

Sodium nitrite (5.07 g, 73.4 mmol, 4.8 eq) was dissolved in water (270 mL) and highly concentrated HCl (6 mL). 6-Bromo-1H-indole (3.0 g, 15.3 mmol, 1.0 eq) dissolved in acetone (75 mL) was slowly added to the aqueous solution. The reaction mixture was stirred for 19 hours. Then, the aqueous layer was extracted using ether (50 mL) and hexane (500 mL). The combined organic layer was washed with water and brine, dried with Mg₂SO₄, filtered, and then concentrated. Purification by column chromatography (20% BtOAc dissolved in hexane) yielded the aldehyde (3) as an orange solid substance (1.7 g; yield=50%).

### 6-Bromo-3-(4-(4-methylpiperazin-1-yl)-1H-benzo[d]imidazol-2-yl)-1H-indazole (4)

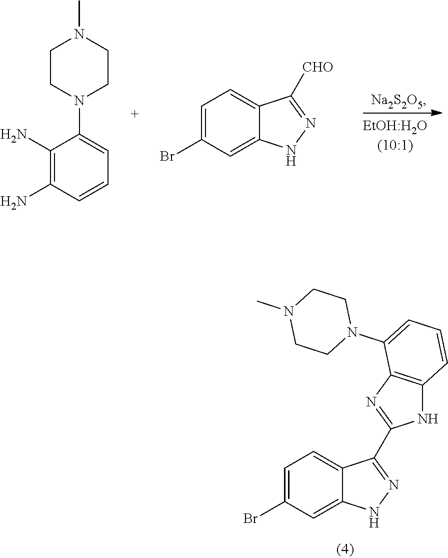

The aldehyde (3, 605 mg, 2.69 mmol) and the diamine (2, 554 mg, 2.69 mmol) were dissolved in ethanol (15 mL), and sodium metabisulfite (306 mg, 1.61 mmol) dissolved in water (2 mL) was added thereto. The reaction mixture was stirred for 17 hours at room temperature. The precipitate was filtered and washed with ethanol. After evaporating the solvent, the residue was washed with methylene chloride. The precipitating solid was dried and obtained as a brown solid substance (4, 500 mg; yield=45.5%).

### Tert-butyl 6-bromo-3-(1-(tert-butoxycarbonyl)-4-(4-methylpiperazin-1-yl)-1H-benzo[d]imidazol-2-yl)-1H-indazole-1-carboxylate (5)

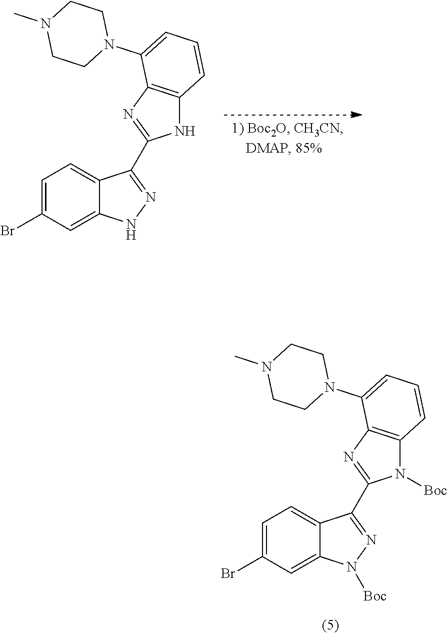

6-Bromo-3-(4-(4-methylpiperazin-1-yl)-1H-benzo[d]imidazol-2-yl)-1H-indazole (4, 4.7 g, 11.59 mmol) was dissolved in acetonitrile (150 mL), and DMAP (5.67 g, 46.39 mmol) and (Boc)₂O (10.124 g, 46.39 mmol) were added. The reaction mixture was stirred for 15 hours at room temperature. After evaporation, the reaction mixture was extracted with methylene chloride and water. The organic layer was washed with brine, dried with Mg₂SO₄, filtered, and then concentrated under reduced pressure. Purification by silica gel column chromatography (MC:MeOH=20:1) yielded a fluorescent solid substance (5, 1.7 g; yield=23.9%).

### Tert-butyl 3-(1-(tert-butoxycarbonyl)-4-(4-methylpiperazin-1-yl)-1H-benzo[d]imidazol-2-yl)-6-(4-(tert-butoxycarbonylamino)phenyl-1H-indazole-1-carboxylate (6a)

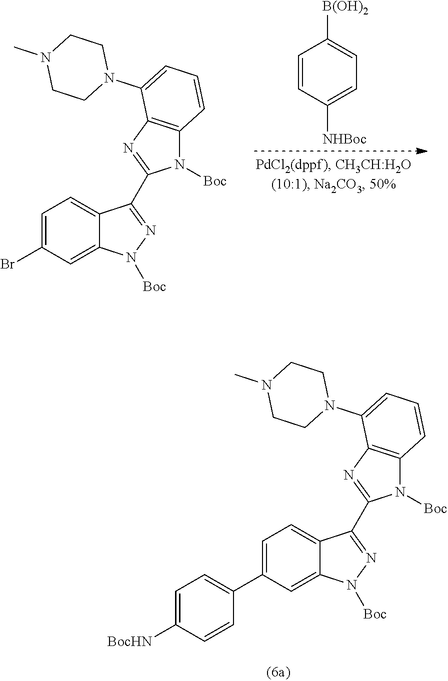

Tert-butyl 6-bromo-3-(1-(tert-butoxycarbonyl)-4-(4-methylpiperazin-1-yl)-1H-benzo[d]imidazol-2-yl)-1H-indazole-1-carboxylate (5, 500 mg, 0.81 mmol) was dissolved in ACN:H₂O (15 mL:1.5 mL), and 4-(tert-butoxycarbonylamino)phenylboronic acid (581 mg, 2.452 mmol), PdCl₂(dppf) (0.3 eq) and Na₂CO₃ (432 mg, 4.085 mol) were added. The reaction mixture was stirred for 15 hours at room temperature. After evaporation, the reaction mixture was extracted with methylene chloride and water. The organic layer was washed with brine, dried with Mg₂SO₄, filtered, and then concentrated under reduced pressure. Purification by silica gel column chromatography (MC:MeOH=30:1) yielded a fluorescent solid substance (6a, 200 mg; yield=33%).

### Tert-butyl 3-(1-(tert-butoxycarbonyl)-4-(4-methylpiperazin-1-yl)-1H-benzo[d]imidazol-2-yl)-6-(4-((tert-butoxycarbonylamino)methyl)phenyl)-1H-indazole-1-carboxylate (6b)

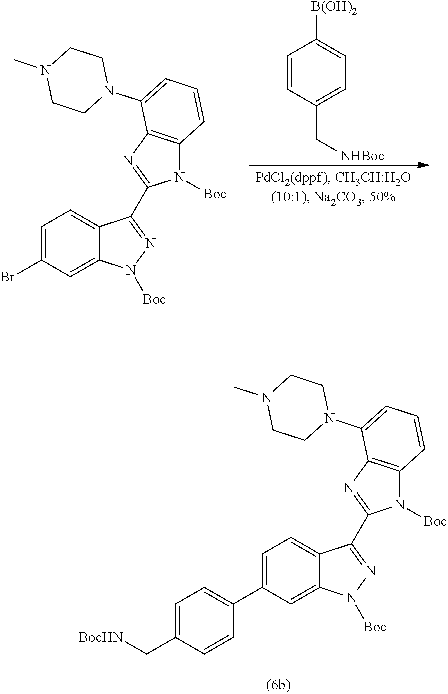

Tert-butyl 6-bromo-3-(1-(tert-butoxycarbonyl)-4-(4-methylpiperazin-1-yl)-1H-benzo[d]imidazol-2-yl)-1H-indazole-1-carboxylate (5, 800 mg, 1.308 mol) was dissolved in ACN:H₂O (20 mL:2 mL), and 4-((tert-butoxycarbonylamino)methyl)phenylboronic acid (985 mg, 3.92 mol), PdCl₂(dppf) (0.3 eq) and Na₂CO₃ (415 mg, 3.924 mol) were added. The reaction mixture was stirred for 15 hours at room temperature. After evaporation, the reaction mixture was extracted with methylene chloride and water. The organic layer was washed with brine, dried with Mg₂SO₄, filtered, and then concentrated under reduced pressure. Purification by silica gel column chromatography (MC:MeOH=30:1) yielded a fluorescent solid substance (6b, 455 mg; yield=48%).

### 4-(3-(4-(4-Methylpiperazin-1-yl)-1H-benzo[d]imidazol-2-yl)-1H-indazol-6-yl)aniline (7a)

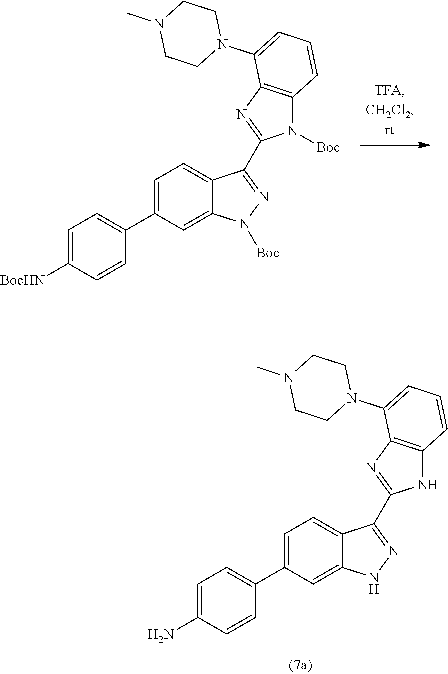

The fluorescent substance (6a, 200 mg) was dissolved in 50% TFA (10 mL), and anisole (1 mL) was added. The reaction mixture was stirred for 4 hours at room temperature and evaporated under reduced pressure. Purification by RP-HPLC (ACN concentration gradient: 20-60%, 30 minutes) yielded a white solid substance (7a, 20 mg).

¹H NMR (CDCl₃, 500 MHz) 8.49 (1H, d, J=7.0), 7.88 (3H, t, J=6.5), 7.66 (1H, dd, J=1.0, 7.0), 7.44 (2H, d, J=7.5), 7.42 (1H, d, J=6.5), 7.36 (1H, t, J=6.5) 6.95 (1H, d, J=6.0), 4.27 (2H, d, J=10.0), 3.75 (2H, d, J=9.5), 3.55 (2H, t, J=10.0), 3.18 (2H, t, J=10.0); MALDI-TOF Mass: 423 (MH⁺).

### (4-(3-(4-(4-Methylpiperazin-1-yl)-1H-benzo[d]imidazol-2-yl)-1H-indazol-6-yl)phenyl)methanamine (7b)

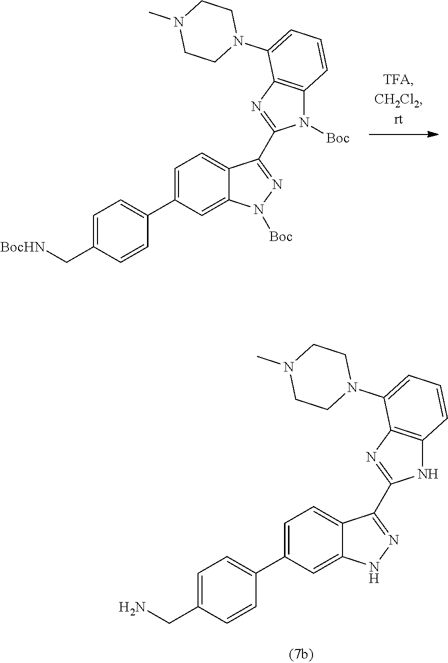

The fluorescent substance (6b, 455 mg) was dissolved in 50% TFA (15 mL)), and anisole (1 mL) was added. The reaction mixture was stirred for 4 hours at room temperature and evaporated under reduced pressure. Purification by RP-HPLC (ACN concentration gradient: 20-60%, 30 minutes) yielded a white solid substance (7b, 100 mg).

¹H NMR (CDCl₃, 500 MHz) 8.50 (1H, d, J=2.5), 7.89 (1H, s), 7.85 (2H, d, J=7.0), 7.66 (1H, dd, J=1.0, 7.0), 7.63 (2H, d. J=7.0), 7.39 (1H, d, J=7.0), 7.34 (1H, t, J=6.5) 6.92 (1H, d, J=6.5), 4.31 (2H, d, J=10.5) 4.22 (2H, s), 3.75 (2H, d, J=10.0), 3.55 (2H, t, J=9.0), 3.27 (2H, t, J=10.5); MALDI-TOF Mass: 437 (MH⁺).

Statistical Analysis

All the experimental results are given as mean±standard deviation. Comparison between the groups was made by Student's t test or one-way analysis of variance (ANOVA). Only the cases where P<0.05 were considered statistically significant.

2. Experimental Results

Effect of PKCζ Inhibitor on Myocardial Contractility

The PKCζ peptide inhibitor inhibits the activity of PKCζ by binding to the PKCζ and interfering with its binding with an activating substrate¹⁵. In order to measure the change in myocardial contractility resulting from the inhibition of PKCζ, the PKCζ inhibitor bound to the TAT peptide which transports extracellular peptides into the cell was used. For comparison, only the TAT peptide or a PKCα inhibitor known to affect myocardial contractility was used.

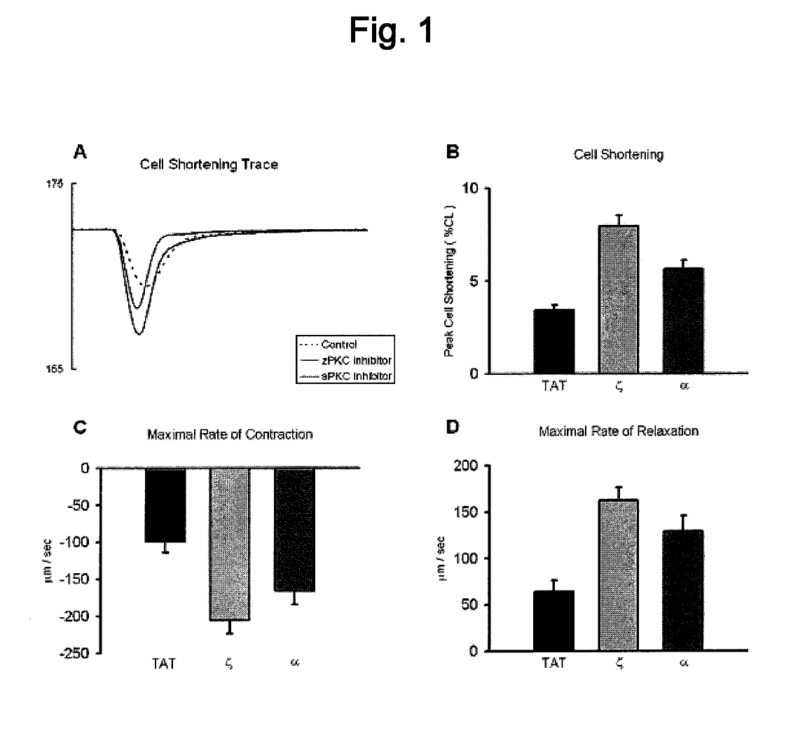

The myocardial contractility measurement revealed that, when treated with the PKCζ inhibitor at 500 nM for 30 minutes, the isolated cardiac myocytes exhibited about 2.4 times increased maximal myocardial contractility as compared to the normal group. The maximal rate of both contraction and relaxation increased by 2 times or more (FIG. 1). This demonstrates that the PKCζ inhibitor enhances myocardial contractility, quickly and potently comparable to other inotropic agents.

Also, the cardiac myocytes with treated with the compounds of Chemical Formula IX and Chemical Formula X at 100 nM for 30 minutes. Treatment with the compound of Chemical Formula IX resulted in about 2.4 times increased maximal myocardial contractility as compared to the normal group. Also, the maximal rate of contraction and relaxation increased by 2 times or more. Treatment with the compound of Chemical Formula X resulted in about 1.4 times increased maximal myocardial contractility as compared to the normal group. The maximal rate of contraction and relaxation increased by 1.2 times.

Study on Inotropic Mechanism of PKCζ Inhibitor

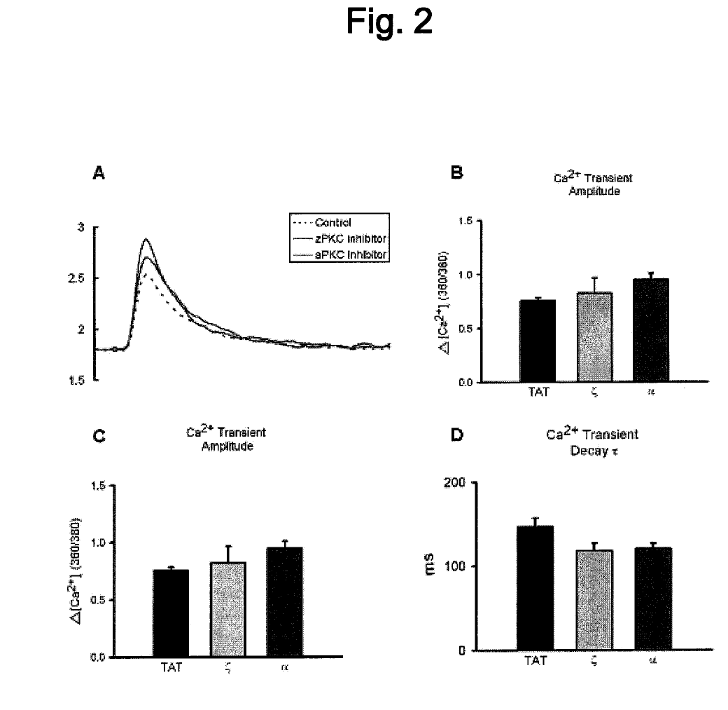

In order to investigate how the PKCζ inhibitor enhances myocardial contractility, change in calcium level and calcium sensitivity of the cardiac myocytes was measured. FIG. 2 shows the change in calcium concentration in the cardiac myocytes caused by the addition of the PKCζ inhibitor. During the relaxation, the calcium concentration of the cells to which the PKCζ inhibitor was added did not show significant difference from the normal group cells. Also, the concentration of calcium released from the sarcoplasmic reticulum during contraction did not show a significant difference. In contrast, for the PKCα inhibitor, a distinct difference of the calcium concentration was observed as compared to the normal group. This result means that the improvement of myocardial contractility by the PKCζ inhibitor is irrelevant of the change of intracellular calcium level.

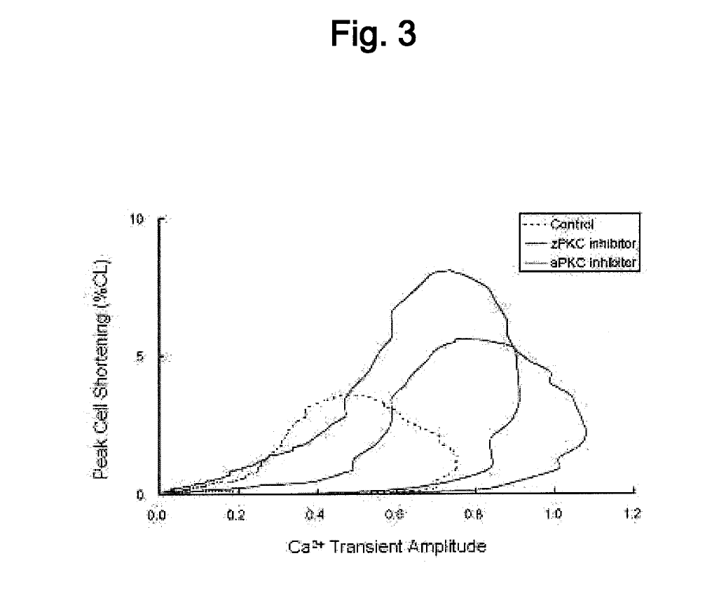

From the hysteresis loops showing change in myocardial contractility caused by change in calcium concentration (FIG. 3), a clear distinctness is found between the PKCζ inhibitor and the PKCα inhibitor. The loop for the PKCα inhibitor is of the same shape as that of the normal group, only with different size. In contrast, the loop for the PKCζ inhibitor is greatly distorted vertically when compared with that of the normal group.

The slope between the maximal contraction and the origin is almost similar for the normal group and the PKCα inhibitor, whereas the PKCζ inhibitor exhibits a steeper slope than the normal group. This suggests that the PKCα inhibitor enhances contractility through increased calcium release from the sarcoplasmic reticulum without change in calcium sensitivity, whereas the PKCζ inhibitor does so by increasing the calcium sensitivity. To conclude, the PKCζ inhibitor enhances myocardial contractility by changing the calcium sensitivity of the cardiac myocytes, which is contrasted with common inotropic agents including the PKCα inhibitor.

Discussion

Treatment of heart failure through enhancing myocardial contractility is the simplest and most fundamental strategy and is attempted by many researches. The experimental results of the present disclosure about the PKCζ inhibitor provide high plausibility of development of new treatment. The results show that treatment of cardiac myocytes with the peptide-type PKCζ inhibitor dramatically enhances myocardial contractility. Also, it was revealed that the PKCζ inhibitor increases calcium sensitivity, differently from existing inotropic agents.

Over the past 10 years, α-adrenergic agonists or PDE III have been used as representative inotropic agents. Although these inotropic agents exhibit distinct increase of myocardial contractility in short time, they aggravate symptoms and increase mortality when used for a long period of time. According to the findings thus far, these adverse effects are caused by the increased oxygen demand of the cardiac muscle, increased apoptosis of the cardiac muscle, and interference with the calcium signal transmitters, resulting in arrhythmia. The calcium sensitivity-increasing inotropic agent is advantageous in that it can improve myocardial contractility without increasing the oxygen demand or the risk of arrhythmia. In this regard, the PKCζ inhibitor that changes calcium sensitivity is considered valuable and promising.

The features and advantages of the present disclosure may be summarized as follows:

(i) The present disclosure provides a composition for preventing or treating heart failure comprising the PKCζ inhibitor as an active ingredient, and a method for screening an agent for treating heart failure.

(ii) Demonstrating for the first time that administration of the PKCζ inhibitor provides inotropic effect by increasing myocardial contractility, the present disclosure will contribute greatly to the prevention or treatment of heart failure.

(iii) Since the present disclosure is based on the change in calcium sensitivity in cardiac myocytes unlike the existing inotropic agents, it can enhance the myocardial contractility without increasing oxygen demand or the risk of arrhythmia.

While the present disclosure has been described with respect to the specific embodiments, it will be apparent to those skilled in the art that various changes and modifications may be made without departing from the spirit and scope of the disclosure as defined in the following claims.

### REFERENCES

[1] Francis G S. Pathophysiology of chronic heart failure. Am. J. Med. 2001; 110: 37S-46S.

[2] Norman R. Alpert, Louis A. Mulieri, David Warshaw. The failing human heart. Cardiovascular Research 54 (2002) 1-10.

[3] Colin D. Mathers, Dejan Loncar. Projections of Global

Mortality and Burden of Disease from 2002 to 2030 PLoS Med. 2006 November; 3(11): e442.

[4] Donald M. Lloyd-Jones, M D, ScM et al. Lifetime Risk for Developing Congestive Heart Failure. Circulation. Dec. 10, 2002; 106(24): 3068-72.

[5] Cowie M R, Mostead A, Wood D A et al. The epidemiology of heart failure. Eur Heart J. 1997; 18: 208-225.

[6] Steven R. Houser, Valentino Piacentino III, Julian Mattiello, Jutta Weisser, and John P. Gaughan. Functional properties of failing human ventricular myocytes. Trends Cardiovasc Med. April 2000; 10(3): 101-7.

[7] G. Michael Felker, M D, and Christopher M. O'Connor, M D Durham, N C. Inotropic therapy for heart failure: An evidence based approach. Am Heart J. September 2001; 142(3): 393-401.

[8] Rockman H A, Chien K R, Choi D J, Iaccarino G, Hunter J J, Ross J Jr, Lefkowitz R J, Koch W J. Expression of a beta-adrenergic receptor kinase 1 inhibitor prevents the development of myocardial failure in gene-targeted mice. Proc Nati Acad Sci USA. Jun. 9, 1998; 95(12): 7000-5.

[9] Jeong D, Cha H, Kim E, Park W J. PICOT inhibits cardiac hypertrophy and enhances ventricular function and cardiomyocyte contractility. Circ Res. 2006; 99: 307-14.

[10] Ohanian V, Ohanian J, Shaw L, Scarth S, Parker P J, Heagerty A M. Identification of protein kinase C isoforms in rat mesenteric small arteries and their possible role in agonist-induced contraction. Circ Res. May 1996; 78(5): 806-12.

[11] Chen L, Hahn H, Wu G, Chen C H, Liron T, Schechtman D, Cavallaro G, Banci L, Guo Y, Bolli R, Dorn G W 2nd, Mochly-Rosen D. Opposing cardioprotective actions and parallel hypertrophic effects of delta PKC and epsilon PKC. Proc Natl Acad Sci. 2001; 98: 11114-9.

[12] Phillipson A, Peterman E E, Taormina P Jr, Harvey M, Brue R J, Atkinson N, Omiyi D, Chukwu U, Young L H. Protein kinase C-zeta inhibition exerts cardioprotective effects in ischemia-reperfusion injury. Am J Physiol Heart Circ Physiol. 2005; 289: H898-907.

[13] Wang J, Bright R, Mochly-Rosen D, Giffard R G. Cell-specific role for epsilon- and betal-protein kinase C isozymes in protecting cortical neurons and astrocytes from ischemia-like injury. Neuropharmacology. 2004; 47: 136-45.

[14] Witte S, Villalba M, Bi K, Liu Y, Isakov N, Altman A. Inhibition of the c-Jun N-terminal kinase/AP-1 and NF-kappaB pathways by PICOT, a novel protein kinase C-interacting protein with a thioredoxin homology domain. J Biol Chem. Jan. 21, 2000; 275(3): 1902-9.

[15] Johnson J A, Gray M O, Chen C H, Mochly-Rosen D. A protein kinase C translocation inhibitor as an isozyme-selective antagonist of cardiac function. J Biol Chem. Oct. 4, 1996; 271(40): 24962-6.

1-15. (canceled)

16. A method for preventing or treating heart failure comprising administering to a subject a protein kinase C ζ (PKCζ) inhibitor.

17. The method according to claim 16, wherein the PKCζ inhibitor is a compound of Chemical Formula I: 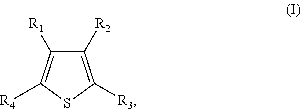
wherein each of R₁ and R₂ is independently alkoxycarbonyl, substituted alkoxycarbonyl, aryl or substituted aryl, wherein at least one of R₁ and R₂ is alkoxycarbonyl or substituted alkoxycarbonyl, and at least one of R₁ and R₂ is aryl or substituted aryl; and each of R₃ and R₄ is independently H, C₁-C₃ alkyl, substituted C₁-C₃alkyl or NHR₅, wherein R₅ is H, 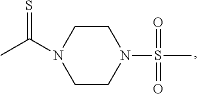
acyl or substituted acyl, and at least one of R₃ and R₄ is NHR₅.

18. The method according to claim 16, wherein the PKCζ inhibitor is a compound selected from the compounds of Chemical Formulas II to VI or a combination thereof:

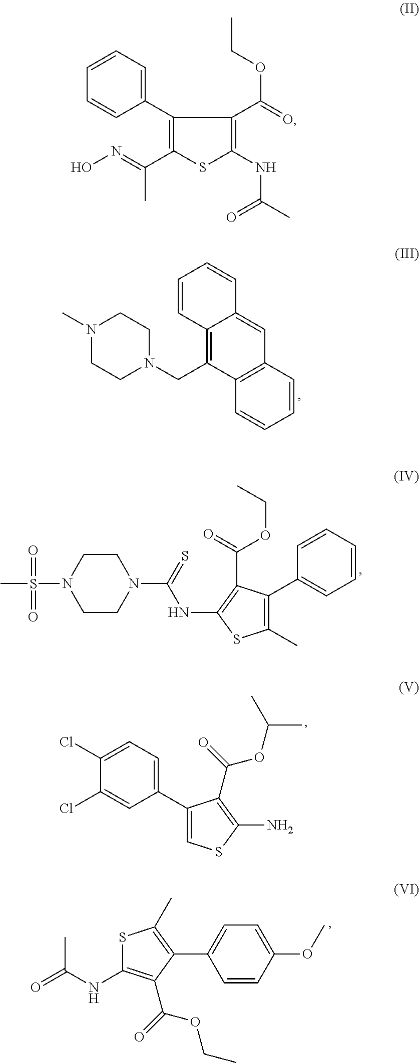

19. The method according to claim 16, wherein the PKCζ inhibitor is a compound of Chemical Formula VII: 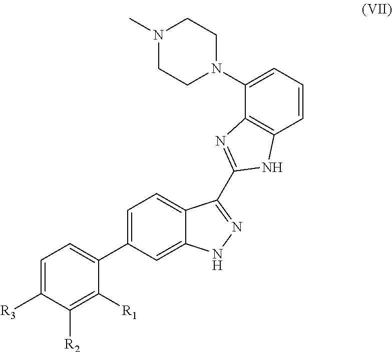
wherein R₁ is hydrogen or C₁-C₁₀ alkoxy, R₂ is hydrogen, halo, amine or C₁-C₁₀ alkoxy, and R₃ is hydrogen, hydroxy, halo, amine, carboxyl, C₁-C₅ alkylamine, C₁-C₅ alcohol, C₁-C₁₀ alkoxy, —NHCO—R₄ (R₄ is C₁-C₅ alkyl), —NH—R₅ (R₅ is C₁-C₅ alkyl), —N(R₆)₂ (R₆ is C₁-C₃ alkyl), —CO—R₇ (R₇ is C₁-C₅ alkyl), —CONH₂ or —SO₂NH₂.

20. The method according to claim 16, wherein the PKCζ inhibitor is a compound of Chemical Formula VIII: 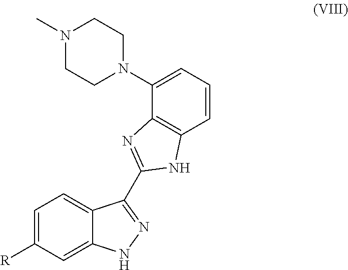
wherein R is indolyl, quinolyl, indazole or benzofuran.

21. The method according to claim 16, wherein the PKCζ inhibitor is a peptide comprising an amino acid sequence of SEQ ID NO: 1 or 2.

22. The method according to claim 21, wherein the peptide is further bonded to a membrane-permeable peptide.

23. The method according to claim 16, wherein the heart failure is induced by cardiac hypertrophy, coronary arteriosclerosis, myocardial infarction, valvular heart disease, hypertension or cardiomyopathy.

24. The method according to claim 16, wherein the PKCζ inhibitor enhances myocardial contractility by increasing calcium sensitivity in cardiac myocytes.

25. A method for screening an agent for treating heart failure, comprising:
contacting a sample to be analyzed with protein kinase C ζ (PKCζ) and
analyzing whether the sample binds to PKCζ or whether the sample inhibits the activity of PKCζ.

26. The method according to claim 25, wherein the heart failure is induced by cardiac hypertrophy, coronary arteriosclerosis, myocardial infarction, valvular heart disease, hypertension or cardiomyopathy.

27. The method according to claim 25, wherein the agent for treating heart failure enhances myocardial contractility by increasing calcium sensitivity in cardiac myocytes.

28. The method according to claim 25, wherein the agent for treating heart failure is an inotropic composition.


SEQUENCE LISTING (2 sequences)

SEQ ID NO: 1 - protein, 17 aa, Artificial Sequence
  Designed peptide based on size and polarity to act as a PKC-zeta inhibitor
SIYRRGARRWRKLYRAN

SEQ ID NO: 2 - protein, 13 aa, Artificial Sequence
  Designed peptide based on size and polarity to act as a PCK-zeta inhibitor
SIYRRGARRWRKL